(19) United States
(12) Patent Application Publication
(10) Pub. No.: US 2005/0064029 A1
(43) Pub. Date: Mar. 24, 2005

(54) Stable pharmaceutical composition

(76) Inventors: Nitin Bhalachandra Dharmadhikari, Mumbai (IN); Sangeeta Vishnu Mirgal, Mumbai (IN)

Correspondence Address: WESTERMAN, HATTORI, DANIELS & ADRIAN, LLP, 1250 CONNECTICUT AVENUE, NW, SUITE 700, WASHINGTON, DC, 20036, US

(21) Appl. No.: 10/910,791

(22) Filed: Aug. 4, 2004

(30) Foreign Application Priority Data
Aug. 4, 2003 (IN) 762/MUM/2003

(51) Int. Cl. A61K 9/20; A61K 31/195

(52) U.S. Cl. 424/464; 514/561

(57) ABSTRACT

The present invention provides a stable pharmaceutical composition comprising gabapentin and pharmaceutically acceptable adjuvants, wherein the composition has an alkaline pH, and wherein at least one of the pharmaceutically acceptable adjuvants is a destabilizer.

The present invention provides a stable pharmaceutical composition of gabapentin.

### BACKGROUND OF THE INVENTION

Gabapentin is a cyclic amino acid, chemically known as 1-(aminomethyl)cyclohexaneacetic acid, and is disclosed in U.S. Pat. Nos. 4,024,175 and 4,087,544. It is a γ-aminobutyric acid (GABA) analog. The patent discloses that the compound may be used for the therapy of certain cerebral diseases, for example, for the treatment of certain forms of epilepsy, faintness attacks, hypokinesia and cranial traumas. Gabapentin is clinically approved as adjunctive therapy in the treatment of partial seizures with and without secondary generalization in patients over 12 years of age with epilepsy. It is also indicated as adjunctive therapy in the treatment of partial seizures in pediatric patients of age 3-12 years, and in the treatment of post-herpetic neuralgia.

The most important problem during the manufacture and storage of gabapentin compositions is the degradation of gabapentin leading to unwanted formation of its corresponding lactam. The lactam formed is toxic and so the limits for the lactam content in gabapentin bulk drug and finished product are specified to be not more than 0.1% (Ref—United States Pharmacopoeia, Pharmacopoeia Forum, Vol 27(5), September-October 2001) and not more than 0.5% (Ref—United States Pharmacopoeia, Pharmacopoeia Forum, Vol 28(2), March-April 2002), respectively. A plethora of prior art addresses the problem of lactam formation and means to control the lactam content in gabapentin formulations.

U.S. Pat. No. 6,054,482 ('482 patent) claims stable and pure pharmaceutical composition in unit dry medicinal dosage form consisting essentially of crystalline, anhydrous gabapentin containing less than 0.5% by weight of its corresponding lactam and less than 20 ppm of an anion of a mineral acid, and one or more pharmaceutically acceptable adjuvants that do not promote conversion of more than 0.2% by weight of the gabapentin to its corresponding lactam form when stored at 25° C. and an atmospheric humidity of 50% for one year. The patent also claims hydroxypropyl methylcellulose (HPMC), polyvinylpyrrolidone (PVP), crospovidone, poloxamer 407 and 188, sodium starch glycolate, copolyvidone, maize starch, cyclodextrin, lactose, talc, copolymers of dimethylamino-methacrylic acid and neutral methacrylic acid ester as adjuvants that may be used with gabapentin without the risk of converting the gabapentin to its corresponding lactam. It teaches that modified maize starch, sodium croscarmellose, glycerol behenic acid ester, methacrylic acid copolymers (types A & C), anion exchangers, titanium dioxide and silica gels like Aerosil 200 destabilize gabapentin and should be avoided in pharmaceutical compositions. The invention requires the use of gabapentin having less than 20 ppm of anion of a mineral acid. Thus, the gabapentin to be used in the composition has to be purified by an ion-exchange. The use of ion-exchange process, when followed by us, proved to be very tedious and time consuming. The method also requires careful monitoring of solution eluting from the resin columns.

PCT application no. WO 99/59572 claims a stabilized solid composition containing γ-aminobutyric acid (GABA) derivative, a humectant, and an auxiliary agent, if necessary. The patent discloses compositions wherein the humectant is dissolved in water or organic solvents and sprayed onto the drug. The patent teaches that the humectant is required for stabilizing gabapentin. PCT application no. WO 99/59573, belonging to the same family, claims and teaches the use of a neutral amino acid as a stabilizer in pharmaceutical compositions comprising gabapentin.

U.S. application No. 20020091159A1 claims a stable pharmaceutical composition comprising one or more amino acids susceptible to lactam formation, one or more stabilizers to inhibit lactam formation, said stabilizer known to reduce ionic activity, and at least 20 ppm of an anion. The patent discloses that compounds, which reduce or inhibit ionic activity due to the high electronegativity and the tendency of contained anions to attract water, may be used as stabilizers. The patent further states that adjuvants used to enhance handling of the formulation may be such that they reduce degradation of amino acids. However, the patent does not disclose stable gabapentin compositions having alkaline pH.

U.S. patent application No. 20020012679A1 discloses a process for manufacturing coated particles of γ-aminobutyric acid analogue having less than 0.5% of lactam, wherein particles of the γ-aminobutyric acid analogue are spray coated with a coating solution comprising at least one polymer in at least one organic solvent. The patent teaches the use of a coat surrounding gabapentin particles for stabilizing gabapentin.

U.S. Pat. No. 6,294,198 claims a pharmaceutical tablet comprising more than 76% by weight of gabapentin, wherein particles of gabapentin are spray-coated with a binder solution, mixed with a disintegrant and a lubricant, and compressed into a tablet. The patent teaches the use of coating with a binder as a means to stabilize gabapentin.

U.S. application No. 20020045662A1 claims a pharmaceutical composition comprising gabapentin initially containing less than 0.5% by weight of a corresponding lactam and having pH in the range of 6.8 to 7.3, wherein after one year of storage at 25° C. and 60% humidity, the conversion of gabapentin to its corresponding lactam does not exceed 0.2% by weight of gabapentin. U.S. Pat. No. 6,531,509 claims a pharmaceutical composition comprising gabapentin initially containing less than 0.5% by weight of the corresponding lactam and having greater than 20 ppm of an anion of a mineral acid, wherein after one year of storage at 25° C. and 60% humidity, the conversion of gabapentin to its corresponding lactam does not exceed 0.2% by weight of gabapentin. In these inventions, stable gabapentin compositions are obtained by using gabapentin having an initial lactam content less than 0.5% and a pH in the range of 6.8 to 7.3.

U.S. application No. 200300119908A1 relates to stable gabapentin compositions comprising at least one salt of a nonacidic cation and an anion of a mineral acid, wherein the composition contains at least 20 ppm of the anion of a mineral acid, based on the amount of gabapentin. The patent further discloses that such compositions may be prepared by adding one or more salts of a nonacidic cation and an anion of a mineral acid to gabapentin. Alternatively, the compositions may be prepared by adding an appropriate amount of the nonacidic cation hydroxide salt to a sample of gabapentin containing more than 20 ppm of chlorides, such that the chlorides form a salt with the nonacidic cation. The patent demonstrates that gabapentin compositions may contain large amounts of anion of a mineral acid and remain stable, provided that the counter-ion to the anion is a nonacidic cation. However, the patent does not disclose stable gabapentin compositions having alkaline pH, nor does it teach gabapentin compositions that are stable in the presence of known destabilizers.

PCT application number WO 04/032905A1 claims a wet granulation method for preparing a stable gabapentin tablet, the wet granulation method comprising forming a mixture by dry mixing of a first portion of a binder with the gabapentin, one or more excipients or a combination of the gabapentin and the one or more excipients; and adding a second portion of the binder to the mixture, wherein the second portion of the binder is in the form of a solution or dispersion. The application teaches that stable gabapentin tablets can be obtained by using the claimed process. However, the application does not disclose stable gabapentin compositions having alkaline pH.

PCT application number WO 04/014356A1 claims compositions comprising gabapentin, a basic compound that is a hydroxide or a salt of a weak acid, and at least one other excipient that is not a hydroxide or a salt of a weak acid. The application provides stable tablet compositions that can be obtained by using gabapentin containing over 20 ppm of an ion of a mineral acid, the stabilizing effect being provided by the basic compound used.

We have now found stable pharmaceutical composition comprising gabapentin and pharmaceutically acceptable adjuvants, wherein said composition has an alkaline pH, and wherein at least one of the pharmaceutically acceptable adjuvants is a destabilizer. The conversion of the gabapentin to its corresponding lactam, during manufacture and storage of the composition, does not exceed 0.4% by weight of the gabapentin.

### OBJECTS OF THE INVENTION

It is an object of the present invention to provide a stable pharmaceutical composition comprising gabapentin.

It is another object of the present invention to provide a pharmaceutical composition comprising gabapentin that can be easily formulated with known adjuvants, including adjuvants known to destabilize gabapentin.

It is another object of the present invention to provide a stable pharmaceutical composition comprising gabapentin, wherein the composition has a pH in the range of pH 7.1 to pH 8.5, and wherein conversion of the gabapentin to its corresponding lactam does not exceed 0.4% by weight of the gabapentin.

It is yet another object of the present invention to provide a stable pharmaceutical composition comprising gabapentin, wherein the composition has an alkaline pH and the conversion of gabapentin to its corresponding lactam does not exceed 0.4% by weight of gabapentin, in the presence of known destabilizers.

### SUMMARY OF THE INVENTION

The present invention provides a stable pharmaceutical composition comprising gabapentin and pharmaceutically acceptable adjuvants, wherein the composition has an alkaline pH, and wherein at least one of the pharmaceutically acceptable adjuvants is a destabilizer.

### DETAILED DESCRIPTION OF THE INVENTION

The present invention relates to a stable pharmaceutical composition comprising gabapentin and pharmaceutically acceptable adjuvants, wherein the composition has an alkaline pH, and wherein at least one of the pharmaceutically acceptable adjuvants is a destabilizer.

The pharmaceutical compositions of the present invention are stable in the presence of known destabilizers such as modified maize starch, sodium croscarmellose, glycerol behenic acid ester, methacrylic acid copolymers (types A & C), anion exchangers, titanium dioxide and silica gels like Aerosil 200. These known destabilizers are either acidic themselves or contain acidic impurities, the acidic character being responsible for the degradation of gabapentin to its corresponding lactam. For example, modified maize starch has a pH between 4.5 to 7.0; glycerol behenic acid ester, commercially available as Compritol 888 ATO, has an acid value of about 4.00, and contains varying amounts of palmitic acid (<3.0%), stearic acid (<5.0%), arachidic acid (<10.0%), behenic acid (>83%), erucic acid (<3.0%) and lignoceric acid (<3.0%); sodium croscarmellose has a pH ranging between pH 5 to pH 7; methacrylic acid copolymers have about 46-50.6% methacrylic acid units; Aerosil 200 has pH typically in the range of pH 3.5 to pH 4.4. We have surprisingly found gabapentin compositions that are stable even in the presence of one or more of these acidic adjuvants.

The stable pharmaceutical compositions of the present invention may be obtained by using pharmaceutically acceptable adjuvants that provide an alkaline pH. Preferably, the alkaline pH is in the range of pH 7.1 to pH 8.5. The pharmaceutically acceptable adjuvants that may be used to provide the required alkaline pH include, but are not limited to, alkali and alkaline earth metal salts such as carbonates of sodium, calcium, potassium; sodium hydrogen carbonate, bicarbonates of sodium, calcium, potassium; alkali salts of citric acid such as trisodium citrate; alkali and alkaline earth salts of phosphoric acid such as disodium hydrogen orthophosphate, dipotassium hydrogen phosphate; and organic bases such as meglumine. One or more of these adjuvants may be used to provide the desired alkaline pH. The adjuvants are selected and used in an amount such that the pH of the composition is in the range of pH 7.1 to pH 8.5. Other pharmaceutically acceptable adjuvants that provide a pH in the range of pH 7.1 to pH 8.5 also fall within the scope of the present invention. For example, celluloses and cellulose derivatives may be used in amounts so as to provide an alkaline pH in the range of pH 7.1 to pH 8.5.

The pH of the compositions is measured by methods known in the pharmacopoeial compendia, such as those cited in United States Pharmacopoeia, Pharmacopoeia Forum, Vol 27(5), September-October 2001, and United States Pharmacopoeia, Pharmacopoeia Forum, Vol 28(2), March-April 2002.

The term “pharmaceutical composition” as used herein includes solid oral dosage forms such as pellets, beads, granules and the like, which may be encapsulated or compressed into tablets. The pellets, beads, granules in turn may be prepared by conventional methods known to a person skilled in the art.

The following examples do not limit the scope of the invention and are used as illustrations.

### EXAMPLE 1

The stable gabapentin composition of the present invention was obtained in the form of capsules as mentioned in Table 1 below.

TABLE 1

| | Quantity | |
|---|---|---|
| Ingredients | (mg/capsule) | |
| Gabapentin | 400.0 | |
| Compactrol (Calcium sulfate) | 141.8 | |
| Starch 1500 (Pregelatinised starch) | 10.0 | |
| Sodium bicarbonate | 4.0 | |
| Compritol 888 ATO (Glyceryl behenic acid) | 8.5 | |
| Magnesium stearate | 5.7 | |
| Fill weight | 570 | mg |

The ingredients were sifted and blended together. The pH of a 2% aqueous solution of this blend was found to be 7.66. The blend was filled in size 0 capsules. The initial lactam content of the blend was found to be 0.05% by weight of gabapentin. The capsules thus obtained were stored at 40° C., 75% relative humidity. The lactam content of the capsules was analyzed at the end of one month, and was found to be 0.05% by weight of gabapentin, i.e. there was no increase in the lactam content of the formulation.

### EXAMPLE 2

Stable gabapentin capsules were obtained as mentioned in Table 2 below.

TABLE 2

| | Quantity | |
|---|---|---|
| Ingredients | (mg/capsule) | |
| Gabapentin (unmilled) | 400.0 | |
| Compactrol (Calcium sulfate) | 141.8 | |
| Starch 1500 (Pregelatinised | 10.0 | |
| starch) | | |
| Calcium carbonate | 4.0 | |
| Compritol 888 ATO (Glyceryl | 8.5 | |
| behenic acid) | | |
| Magnesium stearate | 5.7 | |
| Fill weight | 570 | mg |

The ingredients were sifted and blended together. The pH of a 2% aqueous solution of this blend was found to be 8.06. The blend was filled in size 0 capsules. The initial lactam content of the blend was found to be 0.05% by weight of gabapentin. The capsules thus obtained were stored at 40° C., 75% relative humidity. The lactam content of the capsules was analyzed at the end of one month, and was found to be 0.06% by weight of gabapentin, indicating a stable gabapentin composition.

### EXAMPLE 3

Stable gabapentin tablets were obtained as mentioned in Table 3 below.

TABLE 3

| | Quantity |
|---|---|
| Ingredients | (mg/tablet) |
| Gabapentin | 800.0 |
| Lactose anhydrous (DCL 21) | 217.0 |
| Hydroxypropyl methylcellulose (Methocel E5 Premium) | 45.0 |
| L-Hydroxypropyl cellulose (LH-11) | 50.0 |
| Pregelatinised starch (Starch 1500) | 10.0 |
| Glyceryl behenate (Compritol 888 ATO) | 8.0 |
| Magnesium stearate | 20.0 |
| Opadry White YS-1-7003 | 34.5 |

Gabapentin and lactose anhydrous were dry mixed in a rotating mixer granulator and a binder solution, obtained by suspending Methocel in a mixture of isopropyl alcohol and dichloromethane, was added to it. The blend was granulated and the wet mass was semi-dried in fluidized bed dryer at ambient drying temperature for about 5 minutes. The granules thus obtained were milled and dried again. The dried granules were again milled and mixed with L-hydroxypropyl cellulose, starch, Compritol and magnesium stearate. The lubricated mass was compressed to obtain core tablets. These were then coated with Opadry coating solution to a weight gain on 3% by weight of the core. The pH of a 2% aqueous solution of the coated tablet was 7.67.

### EXAMPLE 4

Stable gabapentin tablets were obtained as mentioned in Table 4 below.

TABLE 4

| | Quantity |
|---|---|
| Ingredients | (mg/tablet) |
| Gabapentin | 800.0 |
| Mannitol | 212.0 |
| Hydroxypropyl cellulose (Klucel - LF) | 30.0 |
| Low substituted hydroxypropyl cellulose (LH-11) | 50.0 |
| Pregelatinised starch (Starch 1500) | 10.0 |
| Glyceryl behenate (Compritol 888 ATO) | 8.0 |
| Talc | 20.0 |
| Magnesium stearate | 20.0 |
| Opadry white YS-1-7003 | 34.50 |

Gabapentin and Mannitol were passed through ASTM #20 sieve and mixed in a rotating mixer granulator. Klucel-LF was suspended in a mixture of isopropyl alcohol and dichloromethane and stirred to obtain a clear solution. This solution was used to granulate the mixture of gabapentin and mannitol. The wet mass thus obtained was granulated by passing through a Clit mill, followed by drying of the granules and milling them. The granules thus obtained were lubricated using a mixture of low substituted hydroxypropyl cellulose, pregelatinised starch, glyceryl behenate, talc and magnesium stearate. The lubricated mass was then compressed to obtain the tablets, which were then coated with Opadry white YS-1-7003. The pH of a 2% aqueous solution of the coated tablet was 7.69.

The tablets had an initial lactam content of 0.03%. The lactam content of the tablets was analyzed at the end of three months of storage at 40° C., 75% relative humidity, and was found to be 0.27% by weight of gabapentin, indicating a stable gabapentin composition.

While the invention has been described by reference to specific embodiments, this was done for purposes of illustration only and should not be construed to limit the spirit or the scope of the invention.

1. A stable pharmaceutical composition comprising gabapentin and pharmaceutically acceptable adjuvants, wherein the composition has an alkaline pH, and wherein at least one of the pharmaceutically acceptable adjuvants is a destabilizer.

2. A pharmaceutical composition as claimed in claim 1, wherein the destabilizer is selected from the group comprising modified maize starch, sodium croscarmellose, glycerol behenic acid ester, methacrylic acid copolymers (types A & C), anion exchangers, titanium dioxide and silica gels like Aerosil 200.

3. A pharmaceutical composition as claimed in claim 1, wherein the composition has a pH in the range of pH 7.1 to pH 8.5.

4. A pharmaceutical composition as claimed in claim 1, wherein the conversion of gabapentin to its corresponding lactam does not exceed 0.4% by weight of gabapentin.

5. A pharmaceutical composition as claimed in claim 1, wherein the gabapentin has an initial lactam content of not more than 0.1% by weight.

6. A pharmaceutical composition as claimed in claim 1, wherein the alkaline pH is provided by one or more adjuvants selected from the group comprising alkali and alkaline earth metal salts, organic bases, celluloses and cellulose derivatives.

7. A pharmaceutical composition as claimed in claim 5, wherein the alkaline pH is provided by alkali and alkaline earth metal salts selected from carbonates of sodium, calcium, potassium; sodium hydrogen carbonate, bicarbonates of sodium, calcium, potassium; alkali salts of citric acid such as trisodium citrate; alkali and alkaline earth salts of phosphoric acid such as disodium hydrogen orthophosphate, dipotassium hydrogen phosphate, and mixtures thereof.
